(19) United States
(12) Patent Application Publication
(10) Pub. No.: US 2021/0134395 A1
(43) Pub. Date: May 6, 2021

(54) EARLY INFECTIOUS DISEASE SIGN DETECTION DEVICE, EARLY INFECTIOUS DISEASE SIGN DETECTION METHOD, AND RECORDING MEDIUM

(71) Applicant: NEC CORPORATION, Tokyo (JP)

(72) Inventors: Masahiro HAYASHITANI, Tokyo (JP); Masahiro KUBO, Tokyo (JP); Shigemi KITAHARA, Tokyo (JP)

(73) Assignee: NEC CORPORATION, Tokyo (JP)

(21) Appl. No.: 17/252,973

(22) PCT Filed: Jun. 18, 2019
(86) PCT No.: PCT/JP2019/023999

§ 371 Date: Dec. 16, 2020

(30) Foreign Application Priority Data
Jun. 18, 2018 (JP) 2018-115634

(51) Int. Cl. G16B 40/00; G01N 33/569; G16H 50/20

(52) U.S. Cl. CPC G16B 40/00; G16H 50/20; G01N 33/56983

(57) ABSTRACT

An early infectious disease sign detection device includes: an early infectious disease sign detection unit that generates early sign information indicating an early sign that a determination target patient is going to develop an infectious disease, by using learning data indicating a result of learning about biological information of a patient who has developed the infectious disease among a plurality of patients, and biological information acquired for the determination target patient; and an action information output unit that outputs action information for the infectious disease for the determination target patient, based on the early sign information.

### TECHNICAL FIELD

The present invention relates to an early infectious disease sign detection device, an early infectious disease sign detection method, and a recording medium.

### BACKGROUND ART

Among patients, those with a brain disorder who are showing severe symptoms may possibly develop an infectious disease. Once an infectious disease develops, the duration of patient's hospitalization is prolonged and this causes a burden on the patient. As a related technique, Patent Document 1 discloses a technique in which values of a plurality of physiological parameters of a patient are received, an index value of an acute lung injury is calculated on the basis of the values, and a representation of the index value is displayed on a display to thereby monitor the patient.

### PRIOR ART DOCUMENTS

### Patent Documents

[Patent Document 1] Japanese Unexamined Patent Application, First Publication No. 2018-14131

### SUMMARY OF THE INVENTION

### Problem to be Solved by the Invention

In the technique mentioned above, there is a demand for a technique capable of promptly predicting the development of an infectious disease in a patient.

An example object of the present invention is to provide an early infectious disease sign detection device, an early infectious disease sign detection method, and a recording medium which solve the problem mentioned above.

### Means for Solving the Problem

According to a first example aspect of the present invention, an early infectious disease sign detection device includes: an early infectious disease sign detection unit that generates early sign information indicating an early sign that a determination target patient is going to develop an infectious disease, by using learning data indicating a result of learning about biological information of a patient who has developed the infectious disease among a plurality of patients, and biological information acquired for the determination target patient; and an action information output unit that outputs action information for the infectious disease for the determination target patient, based on the early sign information.

According to a second example aspect of the present invention, an early infectious disease sign detection method includes: generating early sign information indicating an early sign that a determination target patient is going to develop an infectious disease, by using learning data indicating a result of learning about biological information of a patient who has developed the infectious disease among a plurality of patients, and biological information acquired for the determination target patient; and outputting action information for the infectious disease for the determination target patient, based on the early sign information.

According to a third example aspect of the present invention, a recording medium stores a program which causes a computer of an early infectious disease sign detection device to execute: generating early sign information indicating an early sign that a determination target patient is going to develop an infectious disease, by using learning data indicating a result of learning about biological information of a patient who has developed the infectious disease among a plurality of patients, and biological information acquired for the determination target patient; and outputting action information for the infectious disease for the determination target patient, based on the early sign information.

### Effect of the Invention

According to an example embodiment of the present invention, it is possible to promptly predict whether or not a patient is going to develop an infectious disease.

### BRIEF DESCRIPTION OF THE DRAWINGS

FIG. 1 is a schematic diagram of an early infectious disease sign detection system including an early infectious disease sign detection device according to a first example embodiment of the present invention.

FIG. 2 is a hardware configuration diagram of the early infectious disease sign detection device according to the first example embodiment of the present invention.

FIG. 3 is a functional block diagram of the early infectious disease sign detection device according to the first example embodiment of the present invention.

FIG. 4 is a diagram showing a processing flow of a learning process of the early infectious disease sign detection device according to the first example embodiment of the present invention.

FIG. 5 is a diagram showing a processing flow of an early infectious disease sign detection process of the early infectious disease sign detection device according to the first example embodiment of the present invention.

FIG. 6 is a diagram showing a configuration of an early infectious disease sign detection device according to a second example embodiment of the present invention.

### EXAMPLE EMBODIMENTS

Hereinafter, an early infectious disease sign detection device according to an example embodiment of the present invention will be described, with reference to the drawings.

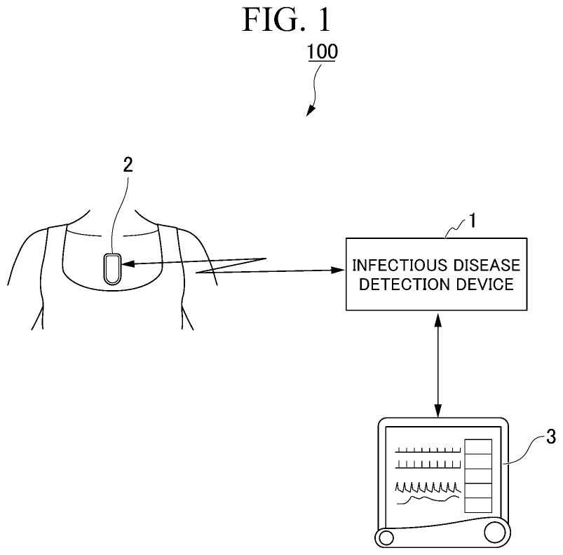

FIG. 1 is a schematic diagram of an early infectious disease sign detection system 100 including an early infectious disease sign detection device 1 according to a first example embodiment of the present invention.

As shown in FIG. 1, the early infectious disease sign detection system 100 includes an early infectious disease sign detection device 1, a measurement device 2, and a display device such as a monitor 3.

The early infectious disease sign detection device 1 is communicatively connected to the measurement device 2 and the monitor 3. The display device may be a terminal other than the monitor 3. For example, the early infectious disease sign detection device 1 may be communicatively connected to a display device such as a terminal carried by a doctor or a nurse.

The early infectious disease sign detection device 1 acquires state information including biological information of a patient from the measurement device 2. The early infectious disease sign detection device 1 may acquire state information of a patient directly inputted by a doctor or a nurse.

The early infectious disease sign detection device 1 outputs the state information, an estimated result of infectious disease development, action information, and so forth to the monitor 3. The biological information which the measurement device 2 can acquire from a patient is the state information including at least the transition of the body temperature of a patient and the transition of the respiratory rate of the patient. The measurement device 2 outputs a temperature to the early infectious disease sign detection device 1 at predetermined intervals. Moreover, the measurement device 2 outputs a respiratory rate per unit time to the early infectious disease sign detection device 1 at predetermined intervals. In addition, the measurement device 2 may detect a pulse rate, an electrocardiographic potential, an acceleration, and so forth and may output them to the early infectious disease sign detection device 1. Furthermore, the measurement device 2 may detect a blood oxygen saturation (SpO₂) and output it to the early infectious disease sign detection device 1.

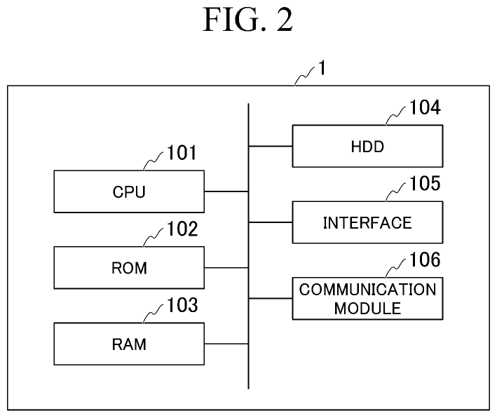

FIG. 2 is a hardware configuration diagram of the early infectious disease sign detection device 1.

The early infectious disease sign detection device 1 is a computer, and, as shown in FIG. 2, includes hardware such as a CPU (Central Processing Unit) 101, a ROM (Read Only Memory) 102, a RAM (Random Access Memory) 103, an HDD (Hard Disk Drive) 104, an interface 105, and a communication module 106.

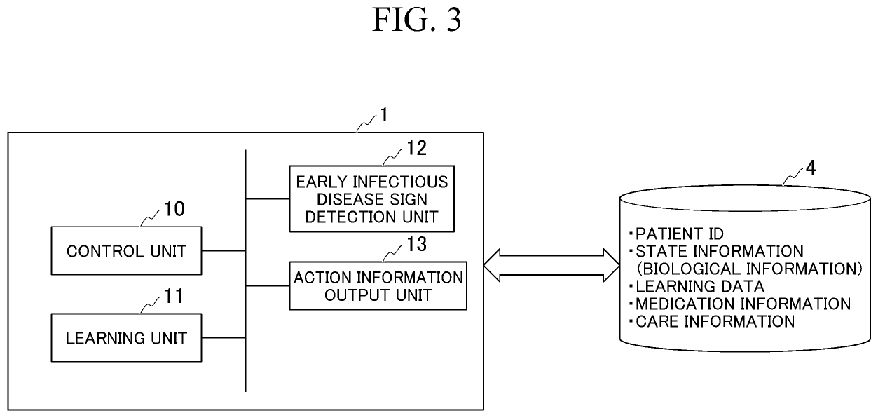

FIG. 3 is a functional block diagram of the early infectious disease sign detection device 1.

As shown in FIG. 3, upon activation, the CPU 101 of the early infectious disease sign detection device 1 executes an early infectious disease sign detection program. As a result, the early infectious disease sign detection device 1 includes functions of a control unit 10, a learning unit 11, an early infectious disease sign detection unit 12, and an action information output unit 13.

The control unit 10 controls the early infectious disease sign detection device 1.

The learning unit 11 performs machine learning on the basis of the state information which includes at least the transition of the body temperature of a patient and the transition of the respiratory rate of the patient, and of an infectious disease development result, to thereby generate learning data. The infectious disease development result may be a result which indicates whether or not an infectious disease has developed. The learning unit 11 may perform machine learning on the basis of the state information which includes at least the transition of the body temperature of an infectious disease patient and the transition of the respiratory rate of the infectious disease patient, to thereby generate learning data.

The early infectious disease sign detection unit 12 generates early sign information indicating an early sign that a determination target patient is going to develop an infectious disease, using learning data indicating a result of learning about biological information of (at least) patients who have developed the infectious disease among patients, and biological information acquired for the determination target patient. The early sign information may be information indicating whether or not an early sign is present, information indicating the degree of an early sign in a manner of either probability or graded evaluation, and so forth. The learning data may be a result of learning about biological information of patients who have developed a certain infectious disease, and about biological information of patients who have not developed the certain infectious disease.

The action information output unit 13 outputs action information for the infectious disease of the determination target patient, on the basis of the early sign information.

The present example embodiment shows an example of a case where the determination target patient is a newly hospitalized patient. Moreover, the present example embodiment shows an example of a case where the patient is a brain disorder patient.

The early infectious disease sign detection device 1 is communicatively connected to a database 4 as shown in FIG. 3. The database 4 stores the state information in association with a patient ID (a patient identification information). Moreover, learning data generated by the learning unit 11, and action information such as medication information and care information corresponding to infectious diseases are recorded in the database 4.

Hereinafter, there is described a process for promptly predicting whether or not a brain disorder patient is going to develop an infectious disease, by means of the processing of the early infectious disease sign detection device 1.

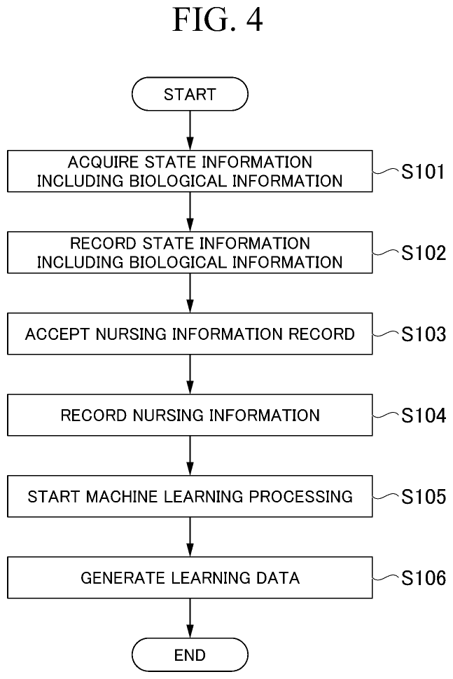

FIG. 4 is a diagram showing a processing flow of a learning process of the early infectious disease sign detection device 1.

First, the early infectious disease sign detection device 1 performs the learning process. As a premise of this learning process, the early infectious disease sign detection device 1 acquires state information including biological information from the measurement device 2 attached to a hospitalized brain disorder patient (Step S101). The early infectious disease sign detection device 1 may acquire biological information and other state information input by a doctor or a nurse. The state information may include biological information such as blood oxygen saturation (SpO2), and state information such as sputum count per unit time. Then the early infectious disease sign detection device 1 records this state information including the biological information in the database 4, in association with a brain disorder patient ID (Step S102).

The early infectious disease sign detection device 1 accepts an input of nursing information about each brain disorder patient from a doctor or a nurse (Step S103). The early infectious disease sign detection device 1 records the nursing information in the database 4 in association with the brain disorder patient ID (Step S104). The nursing information may include, for example, information such as the type of infectious disease and whether or not the infectious disease has developed, the number of days of hospitalization, and the timing at which the presence of the early sign of developing the infectious disease is estimated. The number of days of hospitalization may be a value obtained by counting the number of days since the infectious disease developed, with the date of hospital admission serving as a reference date (the first day). In the situation where these pieces of information are recorded for a plurality of brain disorder patients, the early infectious disease sign detection device 1 accepts an input which instructs to start the processing of machine learning (Step S105).

The learning unit 11 of the early infectious disease sign detection device 1 performs the machine learning processing, using the state information and the nursing information of each brain disorder patient to generate learning data for determining an early sign of developing an infectious disease (Step S106). An early sign detection model configured using the learning data is, for example, a model in which state information of a determination target brain disorder patient serves is an input and information indicating whether or not an early sign of developing an infectious disease is present is an output. Moreover, the early sign detection model configured using the learning data may be a model for further outputting the type of an infectious disease which may actually develop after an early sign of developing an infectious disease is determined as being present. The learning unit 11 records the learning data in the database 4. The learning unit 11 may repeat the learning process at a predetermined timing to update the learning data.

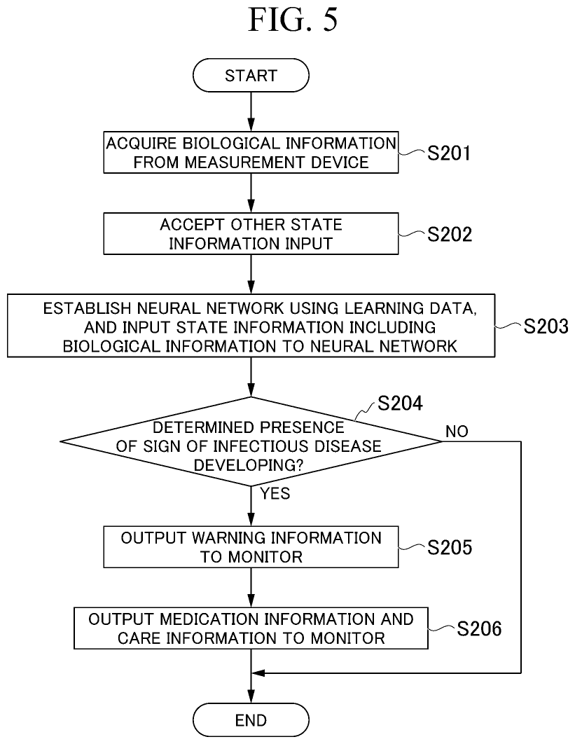

FIG. 5 is a diagram showing a processing flow of an early infectious disease sign detection process of the early infectious disease sign detection device 1.

Next, the early infectious disease sign detection process of the early infectious disease sign detection device 1 will be described.

First, the early infectious disease sign detection device 1 acquires biological information of a newly hospitalized brain disorder patient from the measurement device 2 (Step S201). Furthermore, the early infectious disease sign detection device 1 accepts an input of other state information of the brain disorder patient (Step S202). As described above, the biological information is at least the body temperature and the respiratory rate of the brain disorder patient. Moreover, the biological information and the state information may further include information such as pulse rate, electrocardiographic potential, acceleration, oxygen saturation, and sputum count per unit time.

The early infectious disease sign detection unit 12 establishes the early sign detection model using the learning data recorded in the database 4, and inputs the state information including the biological information to this early sign detection model (Step S203). The early sign detection model may output information indicating whether or not an early sign is present, information indicating the probability of the early sign being present, or information indicating the graded evaluation numerical value of the early sign. The early infectious disease sign detection unit 12 generates early sign information indicating an early sign of an infectious disease on the basis of the state information including the biological information, that is, on the basis of the information output from the early sign detection model. In this example, the early sign information is information indicating whether or not an early sign is present. Then, the early infectious disease sign detection unit 12 determines whether or not there is an early sign of an infectious disease on the basis of the early sign information (Step S204). In the case where the early sign information is information indicating the probability of an early sign, the early infectious disease sign detection unit 12 determines whether the probability is a probability greater than or equal to a predetermined threshold value at which an early sign is determined as being present, and if the probability is greater than or equal to the threshold value, an early sign is determined as being present. In the case where the early sign information is information indicating a graded evaluation numerical value of an early sign, the early infectious disease sign detection unit 12 determines whether the numerical value is greater than or equal to a numerical value indicating a predetermined grade at which an early sign is determined as being present, and if the numerical value is a numerical value greater than or equal to the numerical value of the predetermined grade, an early sign is determined as being present. If an early sign of developing an infectious disease is determined as being present, the early infectious disease sign detection unit 12 outputs to the action information output unit 13, information indicating that there is an early sign of developing an infectious disease. After that, the early infectious disease sign detection unit 12 determines the type of the infectious disease that may be subsequently contracted and outputs it to the action information output unit 13.

Upon acquiring the information indicating the presence of the early sign of developing an infectious disease, the action information output unit 13 outputs warning information to the monitor 3 (Step S205). The warning information is, for example, information for prompting the monitor to output an image indicating the presence of the early sign of developing an infectious disease. As a result, the warning information is output to the monitor 3. A doctor or a nurse can grasp the early sign of developing the infectious disease from the warning information output to the monitor 3.

Also, upon acquiring information on the type of the infectious disease, the action information output unit 13 acquires medication information and care information recorded in the database 4 in association with the type of the infectious disease. The medication information may be information indicating the type of medicine to be administered, the amount of the medicine to be administered, and so forth. The care information may be information indicating an appropriate treatment to be taken for the patient. The action information output unit 13 outputs the medication information and the care information to the monitor 3 (Step S206). This enables a doctor or a nurse to confirm the medication information including the dosage of medicine to be administered and the type of medicine to be administered output to the monitor 3 and administer medicine before the infectious disease develops, and to confirm the care information output to the monitor 3 and provide the patient with an appropriate treatment before the infectious disease develops.

As described above, according to the processing mentioned above, the early infectious disease sign detection device 1 can, before a brain disorder patient develops an infectious disease, determine whether or not an early sign thereof is present. Then, the early infectious disease sign detection device 1 outputs information indicating the presence of the early sign of developing the infectious disease, and thereby can promptly inform a doctor or a nurse of the presence of the early sign of developing the infectious disease. Furthermore, since the early infectious disease sign detection device 1 can output medication information and care information before an infectious disease develops, a doctor or a nurse can administer medicine and provide a treatment in an appropriate, prompt, and correct manner.

In the above description, an example of a case has been described where the presence/absence of an early sign of an infectious disease is determined for a brain disorder patient; however, the early infectious disease sign detection device 1 may determine the presence/absence of an early sign of an infectious disease for other patients. In such a case also, the learning process and the early infectious disease sign detection process are performed similarly.

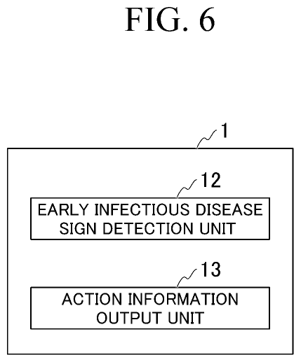

FIG. 6 is a diagram showing a configuration of an early infectious disease sign detection device according to a second example embodiment of the present invention.

It is sufficient that the early infectious disease sign detection device 1 includes at least the early infectious disease sign detection unit 12 and the action information output unit 13.

The early infectious disease sign detection unit 12 determines whether or not there is an early sign that a determination target patient is going to develop an infectious disease, using learning data indicating a result of learning about biological information of patients who have developed the infectious disease among patients, and biological information acquired for the determination target patient.

In the case where an early sign of the determination target patient developing an infectious disease is determined as being present, the action information output unit 13 outputs action information for the infectious disease.

The early infectious disease sign detection device 1 mentioned above has a built-in computer system. The process of each processing described above is stored in a computer-readable recording medium in a form of a program, and the processing mentioned above is performed by a computer reading and executing the program. Here, the computer-readable recording medium refers to a magnetic disk, a magnetic optical disk, a CD-ROM, a DVD-ROM, a semiconductor memory, or the like. Moreover, the computer program may be distributed to a computer via a communication line, and the computer having received the distributed program may execute the program.

Also, this program may be a program for realizing some of the functions described above. Furthermore, the program may be a so-called difference file (a difference program) which can realize the functions described above in combination with a program already recorded in the computer system.

This application is based upon and claims the benefit of priority from Japanese patent application No. 2018-115634, filed June 18, 2018, the disclosure of which is incorporated herein in its entirety by reference.

### INDUSTRIAL APPLICABILITY

The present invention may be applied to an early infectious disease sign detection device, an early infectious disease sign detection method, and a recording medium.

### REFERENCE SYMBOLS

1 Early infectious disease sign detection device
2 Measurement device
3 Monitor
4 Database
10 Control unit
11 Learning unit
12 Early infectious disease sign detection unit
13 Action information output unit

1. An early infectious disease sign detection device comprising:
at least one memory configured to store instructions; and
at least one processor configured to execute the instructions to:
generate early sign information relating to an early sign that a determination target patient is going to develop an infectious disease, by using a result of learning about biological information of a patient who has developed the infectious disease among a plurality of patients, and biological information acquired for the determination target patient; and
output action information for the infectious disease for the determination target patient, based on the early sign information.

2. The early infectious disease sign detection device according to claim 1,
wherein the patient who has developed the infectious disease is a brain disorder patient who has developed the infectious disease, and
the determination target patient is a brain disorder patient.

3. The early infectious disease sign detection device according to claim 1,
wherein the processor is configured to execute the instructions to: perform machine learning based on state information which includes at least a transition of a body temperature of the plurality of patients and a transition of a respiratory rate of the plurality of patients, and a development result of the infectious disease, to generate an early sign detection model.

4. The early infectious disease sign detection device according to claim 3, wherein performing the machine learning comprises performing machine learning based on the state information which further includes at least one of a sputum count of the plurality of patients and a blood oxygen saturation of the plurality of patients to generate the early sign detection model.

5. The early infectious disease sign detection device according to claim 1, wherein the action information includes at least a type of medicine and a dosage of the medicine administered for the infectious disease.

6. The early infectious disease sign detection device according to claim 1,
wherein the early sign information comprises information which indicates whether or not the early sign that the determination target patient is going to develop the infectious disease is present, and
outputting the action information comprises outputting the action information for the infectious disease when the early sign that the determination target patient is going to develop the infectious disease is determined as being present based on the early sign information.

7. The early infectious disease sign detection device according to claim 1,
wherein the early sign information comprises information which indicates a degree of the early sign that the determination target patient is going to develop the infectious disease, and
outputting the action information comprises outputting the action information for the infectious disease, based on the degree of the early sign included in the early sign information.

8. An early infectious disease sign detection method comprising:
generating early sign information relating to an early sign that a determination target patient is going to develop an infectious disease, by using a result of learning about biological information of a patient who has developed the infectious disease among a plurality of patients, and biological information acquired for the determination target patient; and
outputting action information for the infectious disease for the determination target patient, based on the early sign information.

9. A non-transitory computer readable recording medium storing a program which causes a computer of an early infectious disease sign detection device to execute:
generating early sign information relating to an early sign that a determination target patient is going to develop an infectious disease, by using a result of learning about biological information of a patient who has developed the infectious disease among a plurality of patients, and biological information acquired for the determination target patient; and
outputting action information for the infectious disease for the determination target patient, based on the early sign information.
